(19) United States
(12) Patent Application Publication
(10) Pub. No.: US 2022/0005065 A1
(43) Pub. Date: Jan. 6, 2022

(54) SYSTEM AND METHOD TO MANAGE A REWARDS PROGRAM FOR PATIENT TREATMENT PROTOCOLS

(71) Applicant: PREDICTMEDIX INC., Toronto (CA)

(72) Inventor: Rajiv Muradia, Ottawa (CA)

(21) Appl. No.: 17/384,773

(22) Filed: Jul. 25, 2021

Related U.S. Application Data
(60) Provisional application No. 63/048,152, filed on Jul. 5, 2020.

(51) Int. Cl. G06Q 30/02; G16H 20/00; G16H 10/60

(52) U.S. Cl. CPC G06Q 30/0233; G16H 10/60; G16H 20/00

(57) ABSTRACT

System and methods provide for a reward management system for prescribed treatment protocol. The methods and system provide an interface to remotely access, monitor and reward a patient/user for compliance to a prescribed medical treatment by providing reward points. The reward points can be redeemed by the patient for goods and services. The method collects the medical data of the patient using one or more medical devices. The captured medical data can be converted into events based on the medical data and user attributes. The events are transformed with a set of rules into reward points by implementing predefined rules. The reward points are converted into promissory notes, which can be redeemed for tangible goods and/or services.

### RELATED APPLICATIONS

This application is related to, and claims priority to, the following:
1. Provisional Application Ser. No. 63/048,152, filed Jul. 5, 2020.
2. Provisional Application Ser. No. 63/048,131, filed Jul. 5, 2020.
3. Provisional Application Ser. No. 63/058,567, filed Jul. 30, 2020.

The subject matter of the related applications, each in its entirety, is expressly incorporated herein.

### FIELD OF THE INVENTION

The present invention relates to methods and systems that achieve patients to comply with a treatment regimen through rewards for compliance.

### FIELD OF THE INVENTION

The present invention provides a novel method and system of management of a rewards program to achieve compliance with patient treatment programs. More specifically, the system and method of management of a reward program for a patient treatment program provides for reward points to motivate the patient to follow the treatment protocol.

### BACKGROUND OF THE INVENTION

Due to large scale outbreak of various infectious, non-infectious, heredity and other lifestyle diseases, it has become important to ensure that patients strictly follow the treatment protocol to live a healthy life. For example, the pre-diabetic person can be treated through lifestyle management by following a prescribed protocol of diet, exercise, and medicine. Most of the people are though not motivated to strictly follow the prescribed diet, exercise and medicine plan resulting in aggravation of the disease. Likewise, a heart patient can manage to remain in good health by following a treatment protocol of diet and exercise coupled with medicine. By following a strict diet, exercise and medicine plan, the heart patient can reduce the chances of the heart attack. Therefore, it is important to motivate the patient to strictly follow the prescribed protocol by a medical expert to maintain good health.

The novel invention provides for a system and methods to motivate a patient to follow a prescribed protocol for maintaining good health. The disclosed novel system monitors the medical measurement of the patient in real time and accordingly provides events for keeping the patient in good health. The events are associated with rewards and recorded in the database as reward events. The reward events are converted into points. The points are credited into the account of the patient, when the patient follows a prescribed protocol for treatment. The prescribed protocol may simply be the adherence of the patient to reward events.

### SUMMARY OF THE INVENTION

System and methods of the present invention provide an innovative method for remote health care. The method provides an interface for a skilled medical advisor to interact, discuss and coordinate with a patient regarding his/her disease and/or treatment plan. In one embodiment, the system includes a processor and a memory connected with a database which may be external to the system or an internal database to acquire medical data using a measurement system, which is networked to one or more distributed systems and/or databases and comprises patient data. The system aggregates the medical data of one or more patients in real time. The system then transforms the medical data based on one or more transformation rules to a set of rewards associated with events to be provided to one or more patients. The rewards associated with events for one or more patients are stored into a database as reward events. The reward events are further transformed into points by another set of transformation rules. The points can be converted into a promissory note(s) for purchase of tangible goods and/or services.

In another embodiment, a method to reward a patient for compliance with the treatment protocol is provided. The method acquires medical data using a medical measurement system, which is networked to one or more distributed systems comprising patient data. The method aggregates the medical data of one or more patients in real time from different medical measurement systems and/or distributed databases. The method then transforms the medical data based on one or more transformation rules to a set of rewards, which are associated with events. The rewards associated with events for one or more patients are stored into a database as reward events. The reward events are further transformed into points by another set of transformation rules. The points can be converted into a promissory note(s) for purchase of tangible goods and/or services.

In yet another embodiment, a computer program to reward a patient for compliance to treatment protocol is provided. The method acquires medical data using a medical measurement system, which is networked to one or more distributed systems comprising patient data. The method aggregates the medical data of one or more patients in real time from different medical measurement systems and/or distributed databases. The method transforms the medical data based on one or more transformation rules to a set rewards, which are associated with events. The rewards associated with events for one or more patients are stored into a database as reward events. The reward events are further transformed into points by another set of transformation rules. The points can be converted into a promissory note(s) for purchase of tangible goods and/or services.

In other embodiments, the medical measurement system may be a real time record-keeping system of the patient, a health insurance system, a health complaisance system, a patient history management system, a disease management system or some other type of medical record keeping system.

In some embodiments, the event may be an exercise schedule, a prescribed medicine, an emergency treatment plan, a recording of physiological parameters such as, but not limited to, blood pressure readings, sugar level readings, body temperature readings, pulse readings, oximeter readings or some other parameters for monitoring health. In addition, the events may be new medicine prescribed by the doctor, a course correction with occurrence of an event such as sudden rise or fall in one or more physiological parameters or medical compliance parameters.

In some embodiments, the events are associated with rewards. The reward is a positive behavior associated with a treatment protocol or compliance to a treatment protocol. Each event associated with the reward results in reward events. The reward events are non-monetary positive reinforcement toward performance of a specific behavior. The event rewards can be transformed into points (monetary benefits). These monetary benefits can be utilized for purchasing tangible products or can be credited in the patients account to redeem the points against medical services or other purchases.

### DESCRIPTION OF FIGURES

Different embodiments will now be described in detail with reference to the drawings, in which:

FIG. 1 illustrates an overview of a system for a rewards program for patient treatment protocols in accordance with an embodiment of the present invention;

FIG. 2 illustrates different components of the reward management module in accordance with an embodiment of the present invention;

FIG. 3 illustrates a reward management system in accordance with an embodiment of the present invention;

FIG. 4 illustrates the process flow for a rewards program for patient treatment protocols in accordance in an embodiment of the present invention;

### DETAILED DESCRIPTION OF THE INVENTION

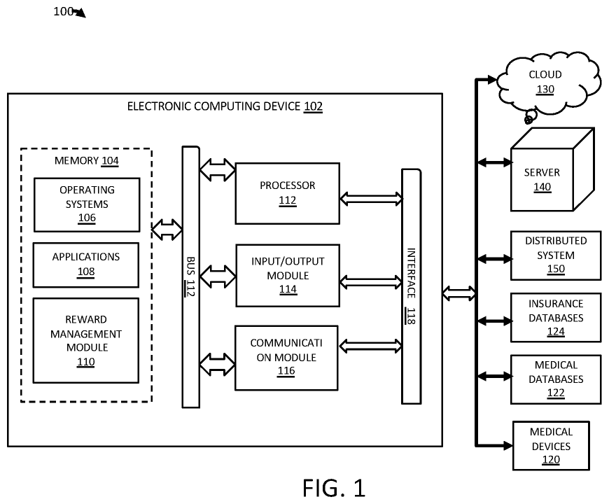

FIG. 1 illustrates the overall environment 100 of the reward management system for patient treatment regime. The environment comprises an electronic computing device 102 networked with a cloud 130, a server 140, a distributed system 150, insurance databases 124, medical databases 122, and medical device(s) 120 for capturing and recording the patient information related to clinical, physical and health related parameters. In some embodiments, the insurance database 124 and/or the medical database 122 may reside on the cloud 130 or the server 140 or the distributed database 150.

The medical devices 120 may be connected with the electronic computing device 102 via wired or wireless network, for example, the medical device 120 can be connected with the electronic computing device 102 by an USB interface or a LAN connection using different type of ports such as micro USB, C type and other type of interface port. In some embodiments, the port may be a physical port or a virtual port. The medical devices 120 may interface via wireless connection with the electronic computing device 102 such as Bluetooth, Edge or Wi-Fi.

The electronic computing device 102 includes a memory 104 comprising operating system 106, one or more applications 108, and a reward management module 110, a processor 112, one or more input/output modules 114, one or more communication modules 116, which are interconnected with each other by an interface bus 112. In addition, the computing device 102 includes an interface 118 to connect with the server 140, the medical database 122 and the cloud 130. Furthermore, the server 140, the medical database 122 and the cloud 130 can be accessed by the reward management module 110 through the internal bus 112 and the interface 118.

The reward management system 110 may be implemented into a single device or may be implemented over a distributed network. Although, in this exemplary embodiment, the reward management system 110 is shown to reside in the memory 104 of the computing device 102, in other embodiments, the reward management module 110 may reside in part in different devices in a network environment.

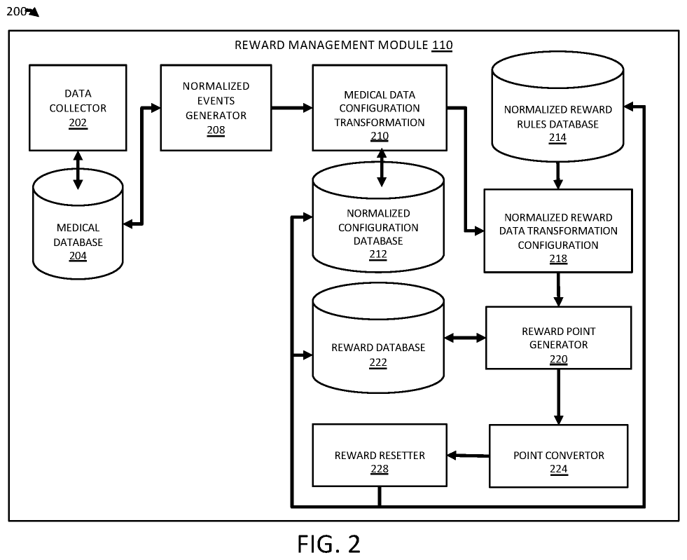

FIG. 2 illustrates different components of the reward management module 110 in an embodiment of the present invention. The reward management module 110 for prescribed treatment of patient treatment comprises a data collector 202, a medical database 204, normalized events generator 208, a medical data configuration transformation module 210, a normalized configuration database 212, a normalized reward rule database 214, a normalized reward data transformation configuration module 218, a reward points generator 220, a reward database 222, a points converter module 224 and a reward resetter module 228.

In an embodiment, the patient is provided with a prescribed plan or a treatment protocol to follow a schedule associated with medical treatment procedures for treatment of aliment(s). The treatment procedure may be related to diet control, exercise plan or taking certain prescribed medicines, taking medical measurements, such as measuring blood pressure, and completing medical questionnaires and other activities related to the treatment protocol.

The reward management system 110 comprises a data collector 202, which may collect medical data having different medical parameters. Each medical parameter may contribute directly or indirectly to the prescribed treatment. Referring to FIG. 2 together with FIG. 1, The medical data/information of the patient may be aggregated using one or more medical devices 120. In some embodiments, the medical data/information may be aggregated from other sources connected directly or indirectly to the reward management module 110. For example, the medical data/information may be aggregated from different sources, such as, medical history databases of the patients, family physician, hospital of the patient, clinical laboratory of the patient, government medical compliance offices, and insurance repositories and databases. The medical data/information aggregated at the data collector 202 may be filtered, processed and transformed to extract relevant information that is related to patient's ailment or aliment(s)/treatment and/or treatment procedure and stored in the medical database 204. Alternatively, the medical database may reside on a server 140 or the cloud 130 or the distributed database 150. The medical database 204 stores the medical data gathered by the data collector 202. In certain embodiments, all collected medical information/data related to patient may be stored in the medical database 204. In some embodiments, the medical information/data stored in the medical database 204 may include all hereditary and genetic information and life history of the patient with respect to medical data comprising medical parameters during life span of the patient. The data stored in the medical database 204 may be designed to be persistent and the data may be structured such that each record is uniquely identifiable. In different embodiments, the medical database 204 may be implemented by using SQL databases such as MySQL, NoSQL or MongoHQ, or key-value stores and file system storage such as a syslog buffer or any other database management scheme or a combination of multiple database management systems.

In some embodiments, the data collector 202 may capture the medical data for the patient from the medical device(s) 120 locally and in other embodiments, remotely using either a wired or a wireless connection. Embodiments may provide multiple ways for data collection both locally as well as remotely.

In another embodiments, the data collector 202 may reside in the cloud 130 or implemented in the server 140 or in a distributed system 150 to collect data remotely while connected by a secure wireless connection. When reward management module 110 is implemented as a software application or module, it may be implemented on a server, a personal computer, a mobile smart computer or web application executed on an electronic computing device, for example, the electronic computing device 102.

In some embodiments, the medical data may be entered manually into the data collector 202, or obtained and uploaded automatically by a medical sensor using wireless technologies such as Wi-Fi, Bluetooth, Edge or others. In this exemplary embodiment, the data collector 202 may be operated by the patient or by a health-care worker acting on behalf of the patient.

The reward management system 110 further includes the normalized (rewards) events generator 208. The normalized events generator 208 receives the medical information/data of the patient from the medical database 204. The normalized events generator 208 takes the data from the medical database 204 and uses configuration process to generate normalized events. The normalized events are generated based on at least medical information/data comprising medical parameters of the patient. In another embodiment, the medical data of the patient may be filtered, processed based on patient attributes in the medical database 204 before passing them to the normalized events generator 208. In embodiments, the patient attributes may be ailments, age, hereditary and non-hereditary disease or some other type of diseases. The process of transformation involves mapping the medical data of the patient by a formula specific to generate normalized events that are included in the prescribed treatment. In some embodiments, the normalized events are captured directly from the medical measurement system associated with the patient, based on the treatment.

In some embodiments, the normalized events generated by the normalized events generator 208 may be prescribed to the patient as part of a prescribed treatment protocol.

In some embodiments, the normalized events may be generated by the normalized events generator 208 based on further inputs provided by a medical expert or a doctor and may be prescribed to the patient as part of a prescribed treatment protocol.

In some embodiments, the normalized events may be generated with continuous intervention of a medical expert. For example, when a patient may be critical or require continuous monitoring or there is a rapidly changing treatment protocol.

The normalized events from the normalized event generator 208 are passed to the medical data configuration transformation 210 for converting the normalized events into normalized event rewards. The medical data configuration transformation 210 is coupled to the normalized configuration database 212. The normalized configuration database 212 contains rules for converting the normalized events into the normalized event rewards. The normalized reward event is based on the medical data record itself and/or optionally data related to the patient. The transformation rules that are stored in the database may be functions which map the medical information/data and patient data by using formulae specific to convert a medical data measurement into a normalized event record.

In some embodiment, the function maybe implemented as a lookup table. The lookup table may be in form of a functional relationship among different variables to predict the normalized reward events from the inputted patient data. In some embodiments, one or more of these variables may include the medical data of the patients, the patient attributes, the DNA profiling information, the historical medical measurements of a healthy and morbid ranges as inputs to arrive at a normalized percentage value between 0 and 100%.

In another embodiment, the function may be implemented by executing certain rules stored in the normalized configuration database 212. The process of transformation is implemented by executing a set of rules stored in the normalized configuration database 212 or alternatively by allowing an expert to manually create customized rules using a user interface.

In some embodiments, the rules for transforming the medical measurements and/or medical data into reward ranges may be determined by medical professionals. For example, a medical professional may decide that a systolic blood pressure of between 115 and 125 is in the healthy range, and a diastolic pressure of between 70 and 85 is in the healthy range. The medical expert may also decide that a blood pressure of 160/110 is extremely dangerous and may accordingly set the rules for rewards.

In some embodiments, the process of transformation implemented with a lookup table may include lookup tables created by a panel of experts and those criteria may be provided in the normalized configuration database 212.

In some embodiments, an additional artificial intelligence module may dynamically determine the reward events based on machine learning trained on such predictions.

The medical data configuration transformation 210 perform the function of converting the medical data of the patient into normalized rewards events. The function provides a relationship between different parameters such as, but not limited to, the medical data of the patient, historical data of the patient, specific ranges of medical data of the patient to create lookup tables. In some embodiments, the specific ranges of the medical data of healthy patient and the specific ranges of medical data of a morbid patient may be used to map the functional relationship of the lookup tables to arrive at event rewards.

In some embodiments, the specific ranges of the medical data of healthy patient and the specific ranges of medical data of a morbid patient may be provided to a machine learning algorithm using artificial intelligence. After the artificial intelligence module has assimilated the learning regarding prediction of reward events, the artificial intelligence module may be utilized for predicting the reward events.

The medical data configuration transformation 210 results are passed to the normalized reward transformation configuration 218. The function of normalized reward transformation configuration 218 is to convert and store normalized rewards events as rewards points. Further, the function maps the time-bounded stream of normalized rewards events per user and per measurement type to quantity of points, where the time boundaries may be part of the configuration. In some embodiments, there may be one quantity of points per user per type of measurement. The function subsequently may combine a set of streams based on other patient-specific configuration information to a scalar which represents the quantity of rewards points.

The normalized reward events may be transformed using the rules provided in the normalized reward rules database 214. In addition, the normalized reward rules database 214 also stores the normalized reward events. In some embodiments, the normalized reward rules database 214 may also store the reward points.

In some embodiments, the normalized reward rules database 214 may include a user interface to allow manipulation of rules for conversion of rules from normalized reward events to reward points.

In some embodiments, the transformation process of normalized reward events to reward points may be a lookup table with the rewards events, user data, measurement type, configuration data, time boundaries, and other configuration data and/or a configured weighting vector for a set of inputs and the output may be a quantity of points.

In some embodiments the transformation process of normalized reward events to reward points may be a computerized data manipulation process using an iterative programming language to manipulate the inputs (rewards events, user data, measurement type, time boundaries, and other configuration data and a weighting vector for a set of inputs) to produce the quantity of points as output.

In some embodiments, the transformation process of normalized reward events to reward points may be a rule engine, which may be driven by stored configured rules with the output being quantity of points. Use of configuration rules for generation of points allows experts in the domain of rewards programs to generate rewards in proportion to the actual value generated.

In some embodiments, the normalized reward rules database 214 stores the normalized rewards events persistently and the data may be structured such that each record is uniquely identifiable.

In embodiments, normalized reward rules database 214 may implemented by using SQL databases such as MySQL, NoSQL or MongoHQ, or key-value stores and file system storage such as a syslog buffer or any other database management scheme or a combination of multiple database management systems.

The reward points are passed from the normalized reward transformation configuration 218 to the reward point generator 220. The reward point generator 220 converts the reward points into normalized points. The normalized points may be a quantity of points.

In some embodiments, the normalized reward points may be directly converted from the normalized reward events.

The normalized reward points are stored in the rewards database 222. The rewards database 222 data may be a persistent database until it is reset by the reward resetter 228.

The normalized reward points generated by the reward point generator 220 is passed to the point convertor 224, which converts the normalized points into promissory notes. In some embodiment, the promissory notes may be equal to some multiple of normalized points, for example, one promissory note may be equal to “k” normalized points, where k is an integer value. In another embodiment, the point convertor 224 may be implemented as a function, which maps normalized points to proportional number of points to a 3rd party rewards engine by using a lookup table. In yet another embodiment, the promissory notes may be equal to some fractional of normalized points, for example, one promissory note may be equal to “k” normalized points, where k is decimal value less than 1.

The reward management module 110 may also include a reward resetter 228, which can be used to reset the patient data to default settings. For example, the reward resetter 228 initializes the patent account information to initial or default values or values defined by the administrator. Further, after the reset procedure has been implemented, the patient data such as reward events, normalized reward events, reward points, normalized reward points and promissory notes are all set to default values. In some embodiments, the default values may be a zero value or values defined by the administrator.

The medical information is processed by server applications to determine how many points to assign to the patient. Eventually the patient is able to transform existing points into a promissory note which can be exchanged for tangible goods and services.

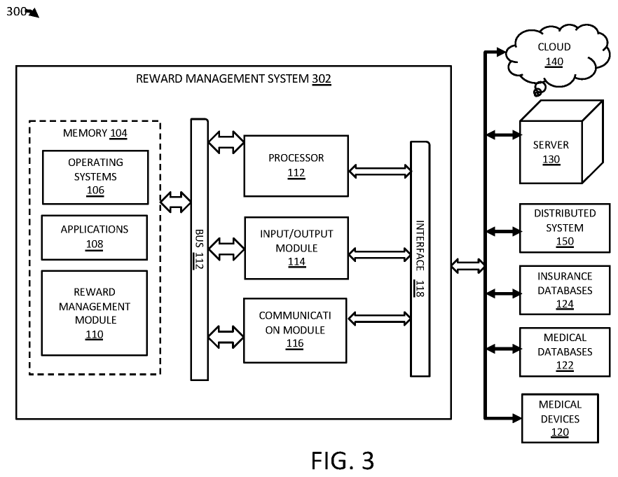

In other embodiments, the reward management module 110 may be implemented as a standalone reward management system 300 as show in FIG. 3. The reward management system 302 is connected by interface bus 118, to one or more servers, systems and distributed hardware such as the cloud 130, the server 140, the distributed system 150, the insurance database 124, the medical database 122, and medical device(s) 120 for capturing and recording patient information related to clinical, physical and health related parameters. In some embodiments, the insurance database 124 and/or the medical database 122 may reside in the on a cloud the server 140 and the distributed database 150.

The reward management system 302 includes the memory 104 comprising the operating system 106, the applications 108, and the reward management module 110, the processor 112, the input/output devices 114, the communication devices 116, which are interconnected with each other by the interface bus 112.

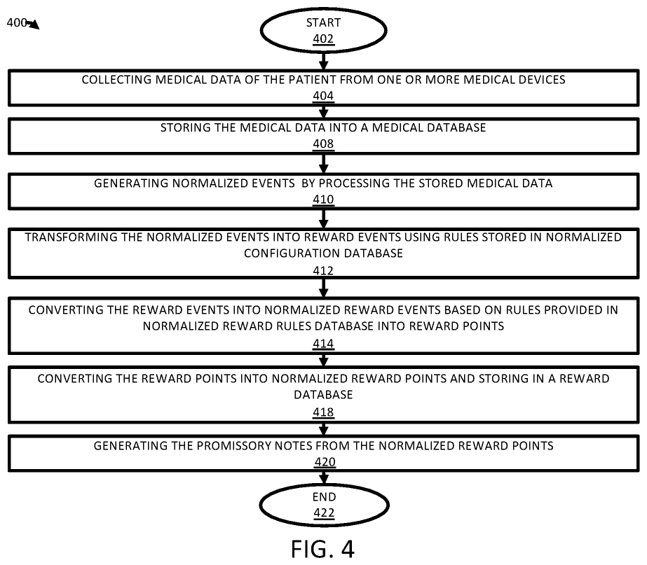

FIG. 4 illustrates the process 400 of the reward management method in an embodiment of the present invention. The process 400 is initiated at step 402 and moves to step 404. At step 404, the reward management process 400 collects the medical data form one or more medical devices. The data collected by one or more medical devices may be medical data of a patient, which further comprises medical parameter of a patient. In some embodiments, the patient may be a normal user of the reward management system. The medical parameters include data of the patient corresponding to clinical reports, body measurements such as temperature, blood pressure and other medical parameters. At step 408, the medical information/data is stored in a medical database. The process 400 may access the stored medical data of the patient/user to generate one or more normalized events related to prescribed treatment of the patient at step 410. The events correspond to the treatment protocol assigned to the patient/user. At next step of transforming 412 the generated events are converted into reward events by using the rules stored in a normalized configuration database. The reward events may also be stored in the normalized configuration database. Next step 414, converts the reward events into normalized reward events using the rules stored in the normalized reward rules database. In some embodiments, the normalized reward events may also be stored persistently in the normalized reward rules database. In some embodiments, the rules for transformation of reward events into normalized reward events may be manually entered by a medical expert or a subject matter expert. In the exemplary embodiment of FIG. 4, the process 400 at step 418 converts the reward point are converted into normalized reward points by using the rules stored in the reward database and the normalized reward points may be stored persistently in the reward database. At step 420, the normalized reward points are converted into promissory notes by a point convertor. In addition, in some embodiments, the process 400 may include a function for resetting and/or redeeming the promissory notes. When the promissory notes are redeemed, the reward resetter may set all of the patient data corresponding to rewards to a default value.

The process 400 may involve monitoring medical information associated with a patient. Typically, a patient is requested to follow a schedule associated with medical procedures, such as, taking a prescription, taking medical measurements, such as measuring blood pressure, and completing medical questionnaires. The medical information is then obtained and stored on the reward management module 110 or the reward management system 302. The medical information is processed in the reward management module 110 or the reward management system 302 applications embedded therein to determine how many points to assign to the patient. Eventually the patient is able to transform existing points into a promissory note which can be exchanged for tangible goods and services.

The data collector 202 may parse a series of messages/data packets. Each data packet or message may have one or more payload messages, for example, each message may have a unique message identifier. In addition, each message may have a device MAC address, which uniquely identifies the medical device that captured the medical data of the patient. The message may have other parameters not limited to a device type such as parameters that may identify the kind of medical device; a timestamp when the data was recorded; a timestamp when the data was saved to the database; a patient identifier which uniquely identifies the patient for whom the data was gathered; a user identifier which uniquely identifies the person who gathered the information, such as a health care worker, physician or the patient himself.

In some embodiments, the message may include the latitude and the longitude of the geographic location where the medical data was gathered or captured. In some embodiments, the message may include some parameters describing the type of message being sent, such as GET requests and DATA. There may be one or more payload records associated with each message. Each payload message comprises a unique payload identifier; a message identifier which uniquely identifies the message to which that payload is attached; the scope of the payload data, which can be a standard measurement type or a proprietary measurement type; a measurement name, which may be names of standard vital measurements including measurements such as pulse, temperature, blood pressure, blood oxygen, blood glucose, weight, and height or proprietary measurements unique to a device manufacturer, a value of the measurement, and the units of the measurement. The data may be stored in any kind of searchable database such as a key-value store or a SQL relational database.

FIG. 3 shows the overview of the environment 300 in which the reward management system 302 operates. As discussed, the reward management system 302 may be implemented as a software or a hardware. When implemented as a hardware, the reward management system 302 may interface with other severs, distributed systems, medical devices and other data points of medical relevance such as insurance companies, government and other medical institutions. The reward management system 300 may be connected to receive data from external medical devices in the network, for example, a remote temperature check may scan the patient for body temperature. The reward management system 300 may monitor one or more patients and continuously capture medical measurements, treatment compliance notifications, and completed medical surveys. The medical information may be captured by a data collector and stored in the medical database via network.

The data collector stores, provides and processes medical measurements, compliance notifications, and completed surveys in a standardized format. The medical database collects medical data and may store the data in a standard format. The normalized configuration database may contain a set of executable rules, which are used to transform medical data into normalized rewards events.

In another embodiment, the normalized rewards event generator applies the executable rules to medical data events. The medical database provides medical data and generates normalized rewards events, which are stored in the normalized events database. The normalized events database and the normalized rewards data configuration transformation is used to accomplish the transformation of a set of normalized rewards events into a quantity of rewards points. Some embodiments may include a computerized rewards points generator, which uses the normalized rewards data transformation configuration to transform normalized rewards events into rewards points. A points converter may convert the accumulated rewards points into promissory notes as a response to a user command. In some embodiments, a rewards resetter, on receiving the notification of completion of conversion of reward points to promissory note may change the normalized events database to render past rewards events unusable for generating points.

An embodiment has been described of a reward management method for a prescribed treatment compliance of a patient, the reward management method comprising: acquiring a medical data of the patient using one or more medical devices, wherein the medical device captures medical data comprising medical parameters, wherein the medical parameters contribute to monitoring of patient condition under a prescribed treatment; storing the different medical parameters of the patient in a medical database; determining the events based on one or more medical parameters; applying configuration rules to determine one or more reward events based on one or more rules stored in the configuration database to generate normalized reward events; processing the one or more normalized reward events to generate reward points based on conversion rules stored in a conversion database, and converting rewards points into promissory notes. The transformation process of the medical data further comprising medical parameters into normalized rewards points furthers includes a data manipulation process determined by medical experts. In embodiments, the conversion of normalized reward events to reward points is driven by a combination of configuration data and a data manipulation process determined by domain experts.

An embodiment has been described of a reward management system for a prescribed treatment compliance of a patient, the reward management system comprising: a data collector having one or more medical devices, wherein the one or more medical devices capture different medical data of the patient comprising medical parameters, the medical parameters contribute to monitoring of patient condition under a prescribed treatment; a medical database for storing the medical data of the patient, wherein the database stores different medical parameters of the patient; a normalized event generator which determines the events for the prescribed treatment compliance of the patient based on the medical data comprising one or more medical parameters; a medical data transformation configuration for converting the events into reward events, wherein the medical data transformation configuration is configured to a normalized configuration database for applying configuration rules to one or more events to generate normalized reward events; a normalized reward rules database for storing the normalized reward events; a normalized reward data transformation configuration implementing functions to generate normalized reward events into reward points; a reward point generator to convert the reward points into normalized points, and a point convertor for converting the normalized points into promissory notes.

We claim:

1. A reward management method for a prescribed treatment compliance of a patient, the reward management method comprising:
acquiring a medical data of the patient using a medical device, wherein the acquired medical data comprises a set of medical parameters, wherein the set of medical parameters reflect patient condition under a prescribed treatment;
storing the set of medical parameters in a medical database;
determining an event based on the set of medical parameters;
applying a set of configuration rules to determine a reward event based on a set of rules stored in a configuration database to generate a normalized reward event;
processing the normalized reward event to generate a reward point based on conversion rules stored in a conversion database, and
converting the rewards point into a promissory note.

2. A method according to claim 1, wherein applying the set of configuration rules to generate the normalized rewards points further comprises a data manipulation process determined by a medical expert.

3. A method according to claim 1, wherein the conversion rules use for processing of the normalized events into the reward point based on conversion rules comprises a combination of a configuration data and a data manipulation process determined by domain experts.

4. A reward management system for a prescribed treatment compliance of a patient, the reward management system comprising:
a data collector having a medical device, wherein the medical device captures a set of medical data of the patient comprising a set of medical parameters, wherein the medical parameters contribute to a monitoring of patient condition under a prescribed treatment;
a medical database for storing the set of medical data of the patient, wherein the database stores different sets of medical parameters of the patient;
a normalized event generator to determine a normalized event for the prescribed treatment compliance of the patient based on the set of medical data comprising the set of medical parameters;
a medical data transformation configuration for converting the normalized event into reward event, wherein the medical data transformation configuration is configured to a normalized configuration database for applying a set of configuration rules to a normalized event to generate a normalized reward event;
a normalized reward rules database for storing the normalized reward event;
a normalized reward data transformation configuration implementing functions to convert the normalized reward event into reward points;
a reward point generator to convert the reward point into normalized reward point, and
a point convertor for converting the normalized reward point into a promissory note.
